(19) United States
(12) Patent Application Publication
(10) Pub. No.: US 2026/0069284 A1
(43) Pub. Date: Mar. 12, 2026

(54) SPINAL MILLING CUTTER, SPINAL MILLING CUTTER SYSTEM, AND METHOD FOR WORKING ON BONE STRUCTURES IN THE REGION OF THE VERTEBRAL COLUMN

(71) Applicant: Joimax GmbH, Karlsruhe (DE)

(72) Inventors: Wolfgang RIES, Linkenheim (DE); Christoph P. HOFSTETTER, Mercer Island, WA (US)

(73) Assignee: Joimax GmbH, Karlsruhe (DE)

(21) Appl. No.: 19/394,474

(22) Filed: Nov. 19, 2025

Related U.S. Application Data
(63) Continuation of application No. PCT/EP2024/063305, filed on May 15, 2024.

(30) Foreign Application Priority Data
May 19, 2023 (DE) 10 2023 113 219.9

(51) Int. Cl. A61B 17/16

(52) U.S. Cl. CPC A61B 17/16; A61B 17/1633; A61B 2017/1602

(57) ABSTRACT

A spinal milling cutter for working on bone structures in the region of the vertebral column. A distal end face of the spinal milling cutter is provided with a milling cutter toothing having a tooth profile with at least two teeth. The spinal milling cutter has an axial passage for receiving a further spinal milling cutter. A vertebral column system is also provided having the spinal milling cutter and having a further spinal milling cutter. A method for working on bone structures in the region of the vertebral column via the spinal milling cutter is also provided.

This nonprovisional application is a continuation of International Application No. PCT/EP2024/063305, which was filed on May 15, 2024, and which claims priority to German Patent Application No. 10 2023 113 219.9, which was filed in Germany on May 19, 2023, and which are both herein incorporated by reference.

### BACKGROUND OF THE INVENTION

### Field of the Invention

The present invention relates to a spinal milling cutter, to a spinal milling cutter system comprising the spinal milling cutter, and to a method for working on bone structures in the region of the vertebral column at a site of use via the spinal milling cutter.

### Description of the Background Art

Spinal milling cutters are known from the prior art. They have a distal end face with a cutting edge for working on bone structures in the region of the vertebral column, such that access is created through the bone structure of a vertebra to an intervertebral disc space.

However, with the known spinal milling cutters, bone structures in the region of the vertebral column can be worked on only in a diameter which remains constant, and which usually corresponds to the outer diameter of the spinal milling cutter. If a change in the working diameter, in particular an increase in it, is desired, the spinal milling cutter initially used must be laboriously removed from the site of use and replaced by a further spinal milling cutter with a larger working diameter.

This significantly extends the procedure time and significantly increases the risk of complications.

### SUMMARY OF THE INVENTION

It is therefore the object of the present invention to propose a spinal milling cutter that enables more flexible and at the same time safer and faster work on the bone structures in the region of the vertebral column.

This object is achieved according to an example of the invention by a spinal milling cutter. This comprises a spinal milling cutter for working on bone structures in the region of the vertebral column, having a distal end face provided with a milling cutter toothing having a tooth profile with at least two teeth, wherein the spinal milling cutter has an axial passage for receiving a further spinal milling cutter. The object of the invention is also achieved by a spinal milling cutter system. The spinal milling cutter system according to the invention has a first spinal milling cutter according to the invention and a second spinal milling cutter which can be accommodated as a further spinal milling cutter in the axial passage of the first spinal milling cutter, and which has a distal end face with a milling cutter toothing having a tooth profile.

Furthermore, the object of the invention is achieved by a method according to example. This method is a method for working on bone structures in the region of the vertebral column at a site of use via a first spinal milling cutter according to the invention, in particular via the spinal milling cutter system according to the invention, and comprises the following steps: inserting the first spinal milling cutter, with a further spinal milling cutter accommodated in the axial passage of the first spinal milling cutter, along a guide into the site of use, wherein the guide is arranged within the axial passage of the first spinal milling cutter, in particular within the axial passage of the further spinal milling cutter, rotating the first spinal milling cutter together with the further spinal milling cutter such that the bone structure at the site of use is worked on along a predefined path, removing the further spinal milling cutter from the site of use, rotating the first spinal milling cutter in such a way that the bone structure is worked on in the desired manner at the site of use, and removing the first spinal milling cutter from the site of use.

A distal direction points to the site of use and a proximal direction points to the surgeon or user of the spinal milling cutter according to the invention. Based upon a three-dimensional, cylindrical coordinate system, an axial direction points parallel to the direction of extension of the spinal milling cutter, and a radial direction points from the axis of extension of the spinal milling cutter perpendicular to this outwards, or from the outside perpendicular to and toward the axis of extension. A direction along the circumference is perpendicular to an axial and a radial direction.

The invention is based upon the fundamental idea that the axial passage of the spinal milling cutter creates a particularly simple structural possibility for accommodating a further spinal milling cutter. In particular during a procedure, the further spinal milling cutter can be guided through the axial passage of the outer spinal milling cutter according to the invention, such that, due to the comparatively smaller outer diameter of the further spinal milling cutter, further, in particular more precise, work can be performed on the bone structure in the region of the vertebral column, essentially seamlessly, at least without the outer spinal milling cutter according to the invention having to be laboriously removed from the site of use. This simplifies and speeds up the procedure, reduces the risk of complications, and also allows for greater flexibility in working on the bone structure.

The invention also makes it possible to first carry out initial work on the bone structure in the region of the vertebral column via the further spinal milling cutter, which is at least partially accommodated in the axial passage of the spinal milling cutter according to the invention and which has a comparatively smaller working diameter, such that the risk of undesired tissue damage is reduced. The further spinal milling cutter is safely guided by the surrounding spinal milling cutter according to the invention. Simultaneously with the use of the further spinal milling cutter, or immediately thereafter, the work on the bone structure can be carried out using the first spinal milling cutter according to the invention with a comparatively larger working diameter, without the further spinal milling cutter having to be removed from the site of use in a time-consuming and complicated manner. The invention therefore enables, in particular, a safer enlargement of the working diameter during the procedure.

Due to the milling cutter toothing on the distal end face of the spinal milling cutter, the work on the bone structure is carried out in a controlled and safe manner, substantially via the distal end face. The spinal milling cutter according to the invention is particularly designed to work on bone structures, such that the spinal milling cutter can cut axially into the bone structure at the site of use. The spinal milling cutter according to the invention can remain in a user-defined position of the bone structure after the work, and thus has a certain hold there.

The further bone milling cutter can then be pushed through the axial passage of the spinal milling cutter according to the invention, and subsequently used as intended in such a way that the further spinal milling cutter continues to work on the bone structure, at least partially within the spinal milling cutter according to the invention, wherein this subsequent work is carried out with a smaller working diameter due to the design. In this way, the spinal milling cutter according to the invention serves as a kind of protective cover and guide for the further spinal milling cutter, accordingly precluding the risk of injury to nerves and soft tissue structures located near the site of use by the further spinal milling cutter, primarily by its toothing, which can in particular have a sharp blade or cutting edge. On the other hand, as already described, the bone structure in the spinal region can first be worked on using the further spinal milling cutter, which is accommodated within the axial passage of the first spinal milling cutter; the first spinal milling cutter serves as a guide for the further spinal milling cutter. After the work on the bone structure using the further spinal milling cutter, further work on the bone structure can be carried out using the first spinal milling cutter, which can be pushed along the further spinal milling cutter to the site of use. This allows a safer increase in the working diameter. In addition, the risk of injury to nerves and soft tissue structures near the site of use due to slippage of the first spinal milling cutter is reduced.

The spinal milling cutter according to the invention can be designed such that the work on the bone structure takes place when the spinal milling cutter rotates about its axis of extension. Preferably, the axis of extension of the spinal milling cutter is arranged within the axial passage. The axial passage can extend over the entire length of the spinal milling cutter, wherein the axial passage can also be referred to as a lumen or cannulation. In an example of the invention, the spinal milling cutter is substantially cylindrical in shape. The spinal milling cutter according to the invention can have an outer diameter between 4 mm and 9 mm, preferably between 7 mm and 8 mm—for example, about 7.3 mm. The spinal milling cutter according to the invention can have a working length between 150 mm and 350 mm, preferably between 220 mm and 250 mm—for example, approximately 235 mm.

Preferably, the tooth profile of the spinal milling cutter is designed as a cutting edge, at least in portions thereof, in order to improve the work on the bone structure. Preferred developments of the spinal milling cutter provide that outer edges of the at least one tooth lie on an outer diameter of the distal end face of the spinal milling cutter and/or that an edge of the at least one tooth taper to a point axially parallel to the distal end face of the spinal milling cutter.

Preferably, the distal end face of the spinal milling cutter has a particularly convex rounding in order to prevent injury to nerves and soft tissue structures in the vicinity of the site of use. In this respect, the rounding can be designed to be atraumatic. Further developments of the invention provide that the distal end face have a concave rounding in order to ensure the cutting ability of the spinal milling cutter, in particular over its entire distal end face. The further spinal milling cutter, which can be accommodated in the axial passage of the spinal milling cutter according to the invention, can also be designed in this way.

Preferably, at least one tooth of the tooth profile is oriented distally and/or radially. Particularly preferably, all teeth of the tooth profile are oriented distally and/or radially. When the spinal milling cutter is used, radially oriented teeth primarily result in work on the bone structure via the lateral surface of the spinal milling cutter, whereas, with distally oriented teeth, the work is more likely to occur via the distal end face of the spinal milling cutter. Further developments of the invention can provide that the spinal milling cutter have two tooth profiles, wherein a first tooth profile has radially oriented teeth and a second tooth profile has distally oriented teeth in order to combine both of the aforementioned advantages. The tooth profiles can be arranged axially offset from one another.

At least one tooth of the tooth profile may not be designed to be mirror-symmetric, wherein the tooth may in particular have two radial elevations with different radial heights. In particular, it is provided that a step-like transition be formed between the radial elevations of a tooth. In the case of multiple radial elevations of a tooth, the profile of the tooth can have multiple steps, thus being designed in a step-like manner. The step-like transition between the radial elevations, especially in a stepped profile, can improve the working due to the resulting sharp edges of the tooth. It can be provided that all teeth not be designed to be mirror-symmetric, in particular that all teeth have two radial elevations each with different radial heights.

Preferably, it is provided that at least one tooth of the tooth profile have a first flank and a second flank, wherein the inclination of the first flank is greater than the inclination of the second flank. The inclinations of the flanks can refer in particular to the axial direction and/or the radial direction. The first flank can therefore be arranged in front of or behind the second flank relative to the direction of rotation of the spinal milling cutter when used as intended.

Preferably, two adjacent teeth of the tooth profile are separated from each other by a parabolic recess, such that the recesses of the tooth profile can receive milled bone structure and ultimately remove it from the site of use.

In an advantageous development of the invention, it can be provided that the spinal milling cutter can have a threaded portion proximal to the milling cutter toothing in order to advantageously enable work on the bone structure via the lateral surface of the spinal milling cutter. In addition, drilling of the bone structure can be carried out in this way, such that the spinal milling cutter is also designed as a spinal drill. This provides the additional advantage that, after the work operation, the spinal drill can be screwed into the bone structure, thus preventing unintentional removal due to tensile forces acting proximally. The threads of the threaded portion also allow the removal of the bone structure that has been worked on. Preferably, the thread of the threaded portion is at least partially designed as a cutting thread.

Preferably, the tooth profile extends proximally in such a way that the threaded portion is formed. The resulting smooth transition between the tooth profile and the threaded portion improves both the working properties of the spinal milling cutter and the removal of the bone structure that has been worked on.

Preferably, the threaded portion has an axial length between 10 mm and 90 mm, preferably between 20 mm and 75 mm, most preferably of about 60 mm.

Preferably, the threaded portion has a thread pitch between 60° and 85°, in particular between 70° and 85°, most preferably between 75° and 80°—for example, 75° or 80°.

In an advantageous development of the invention, it can be provided that the tooth profile can have at least one tooth with a triangular basic shape, wherein the corners can be rounded. At least one tooth of the tooth profile can be designed to be mirror-symmetric. In a further development of the invention, it can be provided that the tip of the tooth, which preferably points distally, be inclined in and/or against the direction of rotation of the spinal milling cutter. Advantageously, all teeth of the tooth profile can point in the distal direction.

Preferably, the distal end face of the spinal milling cutter has a distal projection, wherein the projection is provided in particular with a further tooth profile in order to improve the working properties of the spinal milling cutter in the distal direction via its distal end face. The projection may have an axial length of 1 mm to 5 mm, preferably 3 mm. The projection may be arranged radially centered relative to the longitudinal axis of the spinal milling cutter and/or may have an opening that is designed as part of the axial passage of the spinal milling cutter. The distal projection may have an elliptical, in particular circular, cross-section, wherein the cross-section of the distal projection is smaller than the outer diameter of the spinal milling cutter. Especially if the projection has a wider tooth profile, this reduces the risk of injury to nerves and surrounding soft tissue that are not intended to be worked on.

The tooth profile of the projection can be designed to be mirror-symmetric, wherein it is particularly provided that the tooth profile of the projection have teeth that each have a triangular basic shape. In a further example, the tooth profile may have non-mirror-symmetric teeth, wherein it is preferably provided that the teeth have two radial elevations with different radial heights, between which a step-like transition is formed.

Preferably, a proximal end portion of the spinal milling cutter has a connecting piece which is designed in particular for receiving the further spinal milling cutter. The connecting piece of the spinal milling cutter can be designed in particular for a torque-proof and/or detachable connection to the further spinal milling cutter. Due to the detachable connection, the further spinal milling cutter can be easily removed from the spinal milling cutter according to the invention—for example, as soon as the spinal milling cutter according to the invention has a secure hold in the bone structure being worked on. In particular, in the case of distally oriented teeth on the distal end face of the further spinal milling cutter, the risk of injury to nerves and soft tissue structures in the vicinity of the site of use is eliminated. In an example of the invention, it can be provided that the connecting piece serve as an entrainer for driving the spinal milling cutter. Preferably, the connecting piece has a triangular basic shape in cross-section. For better connectability of the spinal milling cutter according to the invention with the further spinal milling cutter, it can be provided that the connecting piece of the spinal milling cutter according to the invention have an interior space which is designed to be complementary to a connecting piece of the further spinal milling cutter, such that a positive connection possibility is obtained between the spinal milling cutter according to the invention and the further spinal milling cutter. An inner contour of the connecting piece of the spinal milling cutter according to the invention can be designed to be complementary to an outer contour of a connecting piece of the further spinal milling cutter.

The spinal milling cutter system according to the invention can comprise, in addition to the spinal milling cutter according to the invention and the second spinal milling cutter, further instruments for working on the vertebral column. In the sense of the invention, the second spinal milling cutter of the spinal milling cutter system can correspond to the further spinal milling cutter. The second spinal milling cutter may have at least one feature mentioned above in connection with the spinal milling cutter according to the invention. The second spinal milling cutter can be designed as a spinal milling cutter according to the invention. The spinal milling cutter system may include at least one of the following instruments: guide wire, guide rod, guide sleeve, awl, dilator, working cannula, protective sleeve, trocar, needle, hollow needle, Jamshidi needle.

In the spinal milling cutter system according to the invention, the second spinal milling cutter can be detachably connected to the first spinal milling cutter. Preferably, the second spinal milling cutter is connected to the first spinal milling cutter in a particularly torque-proof manner. The second spinal milling cutter can be accommodated in the first spinal milling cutter in a detachable manner. Preferably, the second spinal milling cutter is accommodated in the axial passage of the first spinal milling cutter and/or connected to the first spinal milling cutter. In a further example of the invention, it can be provided that the second spinal milling cutter be able to be fixed axially to the first spinal milling cutter. Preferably, the second spinal milling cutter can be removed from the first spinal milling cutter proximally.

Preferably, the second spinal milling cutter can be connected to the first spinal milling cutter in a torque-proof manner. The second spinal milling cutter can be axially fixed to the first spinal milling cutter in a defined position relative to the first spinal milling cutter, such that the second spinal milling cutter cannot be inadvertently removed from the first spinal milling cutter. The connection between the first spinal milling cutter and the second spinal milling cutter can be designed as an axial lock and/or as an anti-rotation device.

Preferably, the second spinal milling cutter is designed as a further spinal milling cutter according to the invention, such that the second spinal milling cutter also has, in particular, an axial passage for receiving an additional, third spinal milling cutter. The axial passage of the second spinal milling cutter can be designed for receiving a guide, such that the second spinal milling cutter can reach the site of use along the guide. The guide can be designed as an awl and/or as a guide rod and/or as a guide wire. In particular, it can be provided that the distal end face of the second spinal milling cutter have a rounding. In an advantageous development of the invention, it is provided that the rounding be designed to be concave in order to ensure, in particular in combination with distally oriented teeth of the second spinal milling cutter, its cutting ability over the entire distal end face.

Preferably, the second spinal milling cutter has a proximal connecting piece, the outer contour of which is designed in particular to be complementary to the inner contour of the connecting piece of the first spinal milling cutter. A further development provides that the connecting piece of the second spinal milling cutter have a distal connecting piece part, the outer contour of which is designed to correspond to the inner contour of the connecting piece of the first spinal milling cutter. In this way, a structurally simple connection option is provided between the first spinal milling cutter and the second spinal milling cutter. In a further example of the invention, it can be provided that the second spinal milling cutter be able to be driven to rotate, wherein the first spinal milling cutter can be rotated in particular synchronously with the second spinal milling cutter due to a positive connection to the latter.

Preferably, a drive unit can be provided which is designed to drive the first spinal milling cutter and/or the second spinal milling cutter to rotate in order to carry out the desired work on the bone structure at the site of use.

In a further example of the invention, it is provided that the spinal milling cutters be able to be rotated independently of one another. This is particularly advantageous if the first spinal milling cutter is primarily used to work on the bone structure at the site of use over a user-defined path and is then intended to remain in place with a positive connection to the remaining bone structure. The second spinal milling cutter can then be used to further work on the bone structure while the first spinal milling cutter is not rotating, thus avoiding damage to nerve cells and surrounding soft tissue.

Preferably, the second spinal milling cutter has an axial passage which, in particular when connected to the first spinal milling cutter, is arranged concentrically with the axial passage of the first spinal milling cutter. The diameter of the axial passage of the second spinal milling cutter can be adapted in particular with regard to the outer diameter of a guide, such as an awl, an in particular blunt guide rod, or a guide wire, such that the second spinal milling cutter, in particular in combination with the first spinal milling cutter, is provided with safe and reliable guidance to the site of use via the already placed guide. The inner diameter of the axial passage of the first spinal milling cutter can substantially correspond to the outer diameter of the second spinal milling cutter, such that the bone structure which has been worked on is transported out of the site of use via the lateral surface of the outer spinal milling cutter and in particular does not enter a gap between the first spinal milling cutter and the second spinal milling cutter and become lodged there. This reduces the risk of the spinal milling cutters becoming jammed.

The axial passage of the second spinal milling cutter can extend over the entire axial length of the second spinal milling cutter. Preferably, the second spinal milling cutter has an outer diameter between 3 mm and 7 mm, in particular approximately 4 mm. The diameter of the axial passage of the second spinal milling cutter can be between 1 mm and 4 mm, preferably approximately 2.4 mm. The second spinal milling cutter can have a working length between 160 mm and 360 mm, in particular between 230 mm and 280 mm, preferably approximately 256 mm. The axial length of the spinal milling cutter system can correspond to the axial length of the second spinal milling cutter.

Preferably, at least one tooth of the tooth profile of the second spinal milling cutter has a triangular basic shape, wherein a flank of the tooth can have an inclination of approximately 90°. In a further development of the invention, the tooth of the tooth profile has a notch. In particular, it is provided that the at least one tooth be inclined in the direction of or against the direction of rotation of the spinal milling cutter.

Preferably, the tooth profile of the second spinal milling cutter extends proximally such that a threaded portion of the second spinal milling cutter is formed—for example, in that the tooth profile extends proximally in rotation about the axis of extension of the spinal milling cutter. The threaded portion of the second spinal milling cutter can have an axial length between 10 mm and 40 mm, in particular approximately 20 mm. In particular, the threaded portion of the second spinal milling cutter has the same pitch and/or the same direction of rotation as the threaded portion of the first spinal milling cutter. This allows a largely seamless transition to be created between the threaded portion of the second spinal milling cutter and the threaded portion of the first spinal milling cutter, which particularly facilitates the removal of the worked-on bone structure, or also bony structure or bone material.

Preferably, the second spinal milling cutter protrudes from the first spinal milling cutter, in particular its distal end face, by an axial length of between 0.5 mm to 10 mm, in particular between 1.5 mm to 6 mm, preferably by approximately 4 mm.

The spinal milling cutter according to the invention can be designed for working on bone structures in the region of a spinal joint, in particular in the region of an articular process (processus articulares). Preferably, the spinal milling cutter according to the invention is designed to work on the upwardly directed articular process (superior articular process).

The method according to the invention can provide that the further spinal milling cutter be removed from the site of use when the first spinal milling cutter is positively connected to the bone structure being worked on and thus has sufficient hold for further work on the same. Preferably, the spinal milling cutters are inserted into the site of use along an awl, a guide wire, and/or a guide sleeve.

In a further development of the method according to the invention, it can be provided that the arrangement of the guide at the site of use take place via an access to the site of use, wherein the access is created via an access. An awl or a needle, particularly a Jamshidi needle, is used as an access. The access can be created with the help of X-rays and/or navigation, i.e., a navigation device, and/or robotically. The site of use can be in the region of the articular process of a vertebra, in particular in the region of the upwardly directed articular process (superior articular process) of a vertebra. The needle can be cannulated, i.e., have an internal cavity in which the guide can be arranged at the site of use, and in particular can be connected to the bone structure at the site of use. During work on the bone structure at the site of use, the spinal milling cutter, in particular its distal end face and/or its threaded portion, can be surrounded by a protective working sleeve.

Preferably, the guide is connected to the bone structure, in particular anchored to the bone structure, before the spinal milling cutters are inserted into the site of use, in order to ensure improved guidance of the spinal milling cutters to the site of use.

Preferably, before the spinal milling cutter is inserted into the site of use, in particular after the guide has been connected to the bone structure, a protective sleeve, which is also referred to as a working sleeve, is pushed over the guide, in particular over the first spinal milling cutter, to the site of use, in order to protect sensitive tissue at the site of use, in particular nerve structures.

Preferably, the work on the bone structure by the first spinal milling cutter is carried out substantially through its lateral surface, such that the bone structure is worked on primarily laterally, i.e., along the circumference of the spinal milling cutter, in order to protect sensitive structures—for example, nerves. In particular, sensitive nerve structures distal to the spinal milling cutter can be better protected.

Preferably, access to the site of use is via an articular process of a vertebral body, in particular via an upwardly directed articular process of the vertebral body. The method is particularly designed in such a way that the work on the bone structure, in particular in the region of the articular process of a vertebral body, creates access to the spinal canal, or even further into the intervertebral disc space of the vertebral column, in particular to its nucleus pulposus. After the spinal milling cutter has been removed from the site of use, in a further refinement of the method, a working sleeve can be inserted into the intervertebral disc space via the access created by the spinal milling cutter, wherein the nucleus pulposus of the intervertebral disc space can then be removed from it, particularly with endoscopic vision. In this respect, the method can be used in the context of an implantation of an intervertebral implant, which is also referred to as an intervertebral cage or interbody cage.

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The present invention will become more fully understood from the detailed description given hereinbelow and the accompanying drawings which are given by way of illustration only, and thus, are not limitive of the present invention, and wherein:

FIG. 1 shows a spinal milling cutter according to the invention in a perspectival side view,

FIG. 2 shows the spinal milling cutter according to FIG. 1 in a side view,

FIG. 3 shows a further spinal milling cutter, in particular as part of a spinal milling cutter system according to the invention, in a perspectival view,

FIG. 4 shows an enlarged view of the detail G of FIG. 3,

FIG. 5 shows the spinal milling cutter according to FIG. 3 in a side view,

FIG. 6 shows a spinal milling cutter system in a perspectival view,

FIG. 7 shows an enlarged view of the detail C of FIG. 6,

FIG. 8 shows the spinal milling cutter system of FIG. 6 in a further perspectival view,

FIG. 9 shows an enlarged view of the detail H of FIG. 8,

FIG. 10 shows the spinal milling cutter system of FIG. 6 in a side view,

FIG. 11 shows the spinal milling cutter system of FIG. 6 with the further spinal milling cutter fully inserted, in a perspectival view,

FIG. 12 shows an enlarged view of the detail B of FIG. 11,

FIG. 13 shows an enlarged view of the detail A of FIG. 11,

FIG. 14 shows the distal end face according to FIG. 13 in a side view,

FIG. 15 shows a further example of the distal end face according to the detail A of FIG. 11,

FIG. 16 shows the distal end face according to FIG. 15 in a side view,

FIG. 17 shows a further example of the distal end face according to the detail A of FIG. 11,

FIG. 18 shows the distal end face according to FIG. 17 in a side view,

FIG. 19 shows the spinal milling cutter system according to FIG. 11 in a side view,

FIGS. 20-33 show an example of a sequence of a method according to the invention, and

FIGS. 34-45 show an example of a sequence of a further method according to the invention.

### DETAILED DESCRIPTION

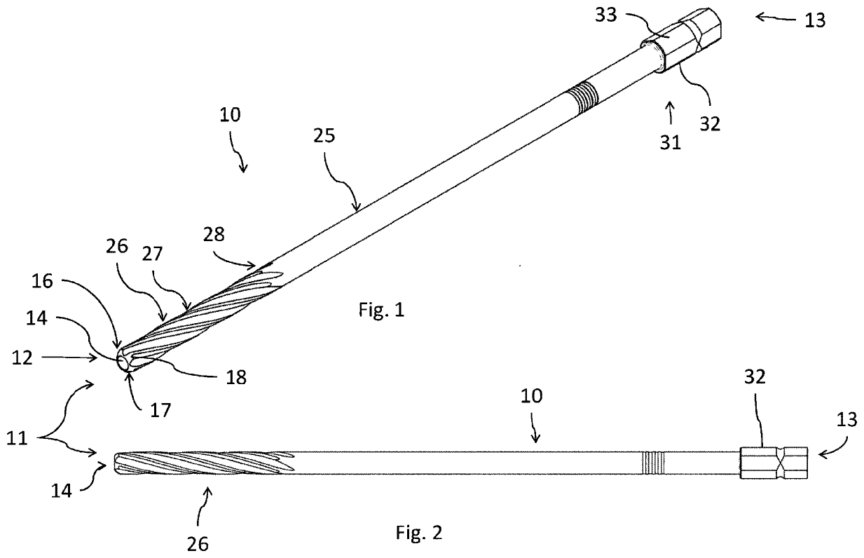

FIG. 1 shows a spinal milling cutter 10 according to the invention for working on bone structures in the region of the vertebral column at a site of use in a perspectival view. The spinal milling cutter 10 has a working length of 235 mm and an outer diameter of approximately 7.3 mm.

On its distal end face 11, the spinal milling cutter 10 has a convex rounding 12, which is also referred to as an atraumatic rounding, since this avoids the risk of unintentional damage to nerves or soft tissue structures located around the site of use when the bone structure is worked on. From the distal end face 11 to the proximal end face 13 of the spinal milling cutter 10, and therefore over its entire axial length of approximately 278 mm, there extends an axially and radially centered passage 14 which is designed for receiving a further spinal milling cutter 15. The passage 14 is circular in cross-section and has a diameter of approximately 4.1 mm.

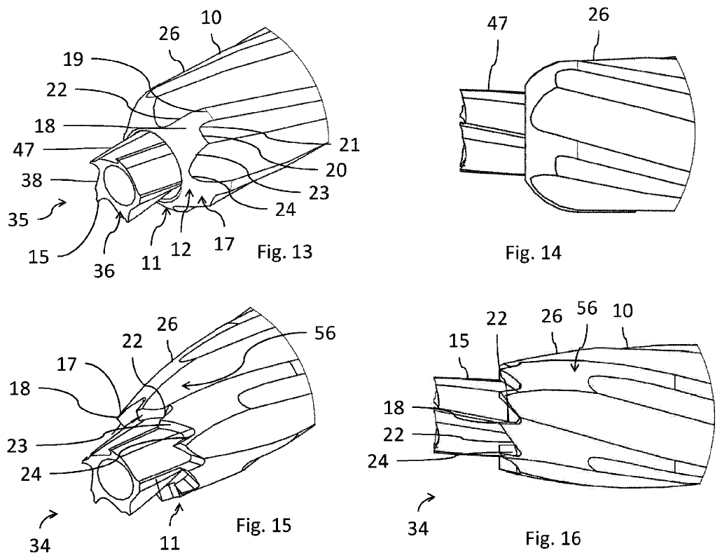

For working on the bone structure, the distal end face 11 of the spinal milling cutter 10 has a milling cutter toothing 16 having a tooth profile 17, wherein the tooth profile 17 in the example shown in FIG. 1 comprises a plurality of radially outward-pointing teeth 18 arranged on the radial outer side of the distal end face 11 and distributed over its circumference. As can be seen in particular from the enlarged view of FIG. 13, each tooth 18 of the tooth profile 17 has two radial elevations 19, 20 with different radial heights, wherein a step-like transition 21 is formed between the radial elevations 19, 20. In a distal view of the distal end face 11, the first radial elevation 19 of a tooth 18 in the circumferential direction has a greater radial height than the second radial elevation 20 of the same tooth 18. The left flank 22 of the tooth 18, associated with the first radial elevation 19, has a higher angle of inclination than the right flank 23, associated with the second radial elevation 20, of the same tooth 18. The radial outer profiles of the teeth 18, which correspond to the tooth profile 17, are designed as cutting edges. Between each two adjacent teeth 18, a recess 24 which is parabolic in the circumferential direction is formed, and connects the right flank 23 of one tooth 18 with the left flank 22 of the next tooth 18 in a stepless manner.

The tooth profile 17 of the distal end face 11 extends proximally such that the lateral surface 25 proximal to the distal end face 11 of the spinal milling cutter 10 is provided with a threaded portion 26 according to FIG. 1. As a result, the outer contour 27 of a cross-section through the threaded portion 26 corresponds at a certain axial height to the tooth profile 17 of the distal end face 11, and is rotated relative thereto by a certain angle about the axis of extension of the spinal milling cutter 10. The threaded portion 26 has an axial length of approximately 60 mm and an angle of inclination of 80°, wherein the threaded portion 26 at its proximal end 28 transitions seamlessly into the substantially smooth lateral surface 25 there.

Proximal to the threaded portion 26, the spinal milling cutter 10 has, on a proximal end portion 31, a connecting piece 32, which is designed for receiving a further spinal milling cutter 15, in particular for a detachable and positive connection to the further spinal milling cutter 15, which will be described further below. The connecting piece 32 has a triangular basic shape with rounded corners 33 and, in the present example, serves as an entrainer to rotate the spinal milling cutter 10 according to the invention synchronously with the further spinal milling cutter 15. FIG. 2 shows the spinal milling cutter according to the invention according to FIG. 1 in a side view, from which in particular the axial length of the spinal milling cutter 10 as a whole as well as of the threaded portion 26 can be seen.

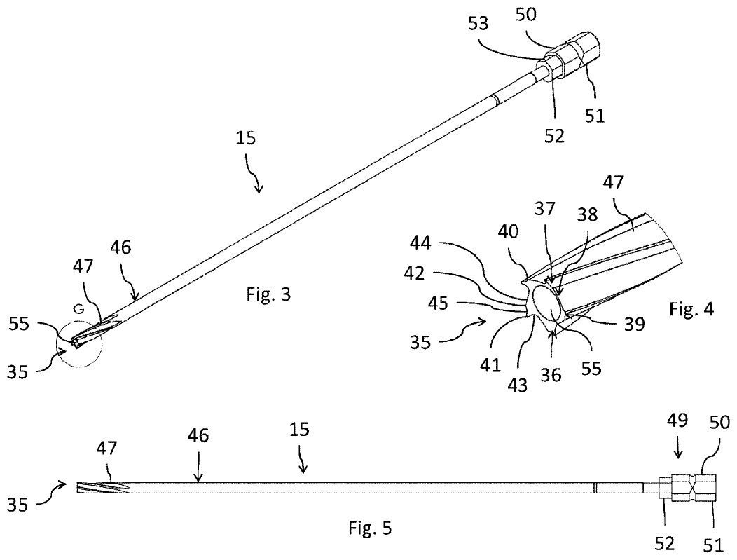

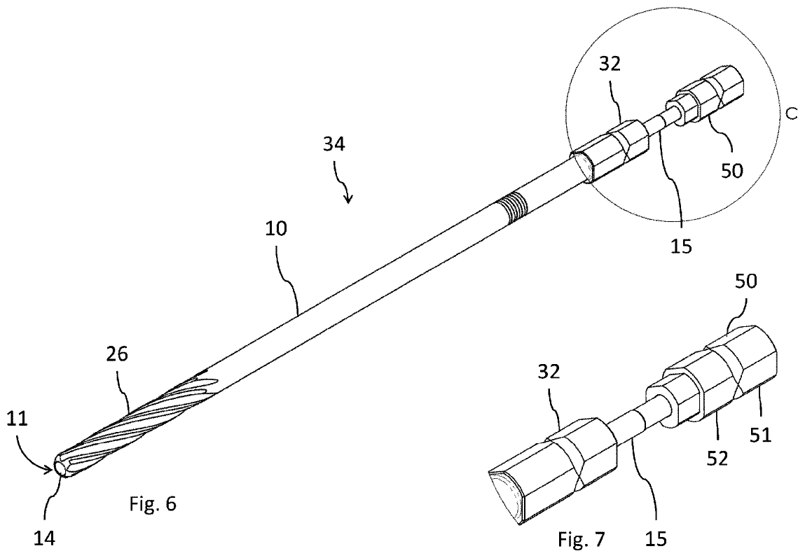

FIG. 3 shows the further spinal milling cutter 15, which, together with the spinal milling cutter 10 according to the invention, e.g., in the example according to FIG. 1, forms a spinal milling cutter system 34 according to the invention, which is shown in FIG. 6. The further spinal milling cutter 15 has a total length of approximately 278 mm, a working length of approximately 255 mm, and an outer diameter of approximately 4 mm, such that the outer diameter substantially corresponds to the inner diameter of the axial passage 14 of the spinal milling cutter 10 according to the invention, wherein at most a comparatively small gap is formed within the axial passage 14 of the spinal milling cutter 10 according to the invention to ensure the mobility of the further spinal milling cutter 15. The further, second spinal milling cutter 15 can thus be accommodated in the axial passage 14 of the first spinal milling cutter 10 according to the invention.

The distal end face 35 of the second spinal milling cutter 15 has, according to the enlarged view of FIG. 4, a concave rounding 36, in contrast to the convex rounding 12 of the first spinal milling cutter 10 according to FIG. 1. A milling cutter toothing 38 with a tooth profile having five teeth 40 is arranged on the circumferential contour 37 of the distal end face 35. The teeth 40 each point distally in the axial direction and outwards in the radial direction. From the enlarged view of the tooth profile 39 according to FIG. 4, it can be seen that the teeth 40 are not mirror-symmetric. The teeth 40 each have two radial elevations 41, 42 with different radial heights, wherein, in contrast to the tooth profile 17 of the first spinal milling cutter 10 according to FIG. 1, the first, left flank 43 of a tooth 40 has an inclination of approximately 90°, while the second, right flank 44 of the tooth 40 has an inclination of approximately 40°. A substantially stepless transition 45 is formed between the radial elevations of a tooth, as well as between two adjacent teeth 40.

Similarly to the first spinal milling cutter 10, the tooth profile 39 of the second spinal milling cutter 15 extends proximally such that the lateral surface 46 of the second spinal milling cutter 15 is provided with a threaded portion 47. The threaded portion 47 of the second spinal milling cutter 15 has an axial length of 20 mm and a thread pitch of 80°, which is also evident in the side view of the second spinal milling cutter 15 according to FIG. 5. The axial length of the threaded portion 47 of the second spinal milling cutter 15 is less than the axial length of the threaded portion 26 of the first spinal milling cutter 10.

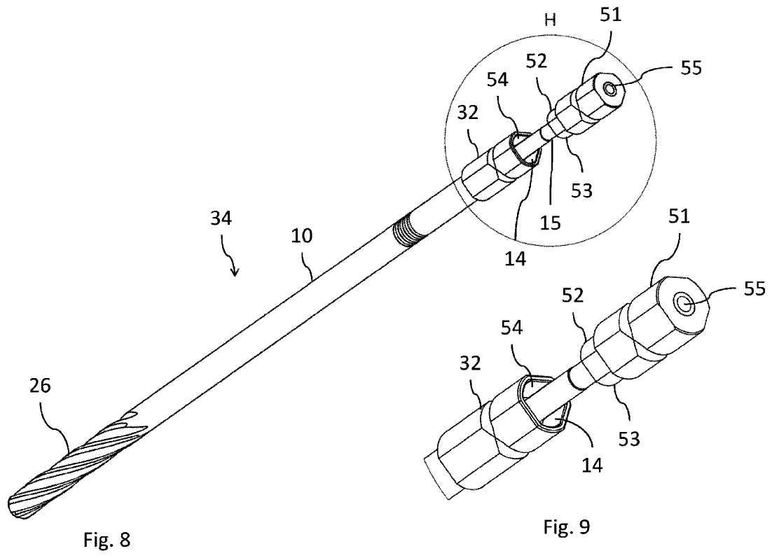

In a proximal end portion 49, the second spinal milling cutter 15 has a connecting piece 50 with a proximal connecting piece part 51 and a distal connecting piece part 52. The distal connecting piece part 52 is designed with its outer contour 53 to correspond to the inner contour 54 of the connecting piece 32 of the first spinal milling cutter 10 shown in FIGS. 8 and 9, such that the second spinal milling cutter 15 can be pushed from the proximal end through the axial passage 14 of the first spinal milling cutter until the distal connecting piece part 52 of the second spinal milling cutter 15 is completely accommodated by the connecting piece 32 of the first spinal milling cutter 10. As such, the second spinal milling cutter 15 can be connected to the first spinal milling cutter 10 in a positive manner, wherein, in this state, via the positive connection thus established, the first spinal milling cutter 10 can be rotated synchronously with the second spinal milling cutter 15 when the latter is driven to rotate. Due to the already mentioned example of the outer contour 53 of the distal connecting piece part 52 corresponding to the inner contour 54 of the connecting piece 32 of the first spinal milling cutter 10, a stepless transition is formed between the proximal connecting piece part 51 of the second spinal milling cutter 15 and the connecting piece 32 of the first spinal milling cutter 10 when the spinal milling cutters 10, 15 are connected to one another, which is also shown in FIGS. 11 and 12.

To drive the second spinal milling cutter 15, a drive device can be connected to the proximal connecting piece part 51 and set it in rotation. Due to the positive connection to the first spinal milling cutter 10, this is then also set into rotation synchronously with the second spinal milling cutter 15. To release the connection, the second spinal milling cutter 15 can be moved proximally relative to the first spinal milling cutter 10 and pushed out of it. According to FIG. 3 and FIG. 4, the second spinal milling cutter 15 has an axial passage 55 with a diameter of approximately 2.4 mm, which is designed for receiving a guide wire 69, an awl 64, or a guide rod 70 as a guide 65 leading to the site of use.

FIG. 6 shows a spinal milling cutter system 34 according to the invention, having the first spinal milling cutter 10 and the second spinal milling cutter 15, in a perspectival view obliquely from the distal end, wherein the second spinal milling cutter 15 is only partially inserted into the axial passage 14 of the first spinal milling cutter 10, coming from the proximal direction. In this position, the distal end face 35 of the second, inner spinal milling cutter 15 does not protrude beyond the distal end face 11 of the first, outer spinal milling cutter, but is, rather, covered by the first spinal milling cutter 10. According to FIG. 7, which is an enlargement of the detail C of FIG. 6, the proximal connecting piece 32 of the first spinal milling cutter 10 is designed for receiving the distal connecting piece part 52 of the connecting piece 50 of the second spinal milling cutter 15.

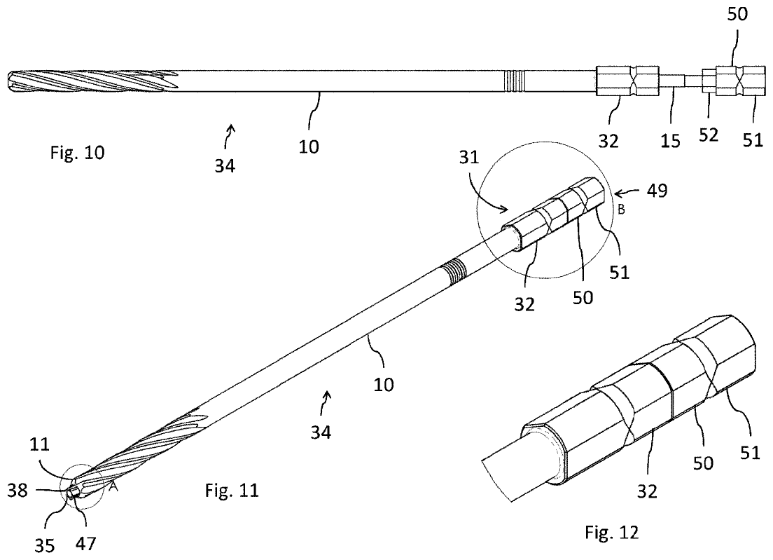

FIG. 8 shows the spinal milling cutter system 34 according to FIG. 6 in a further perspectival view, viz., from an oblique proximal perspective, which shows in particular the axial passage 14 and the inner contour 54 of the connecting piece 32 of the first spinal milling cutter 10, which, as already mentioned, is designed to positively accommodate the distal connecting piece part 52 of the second spinal milling cutter 15. In addition, the axial passage 55 of the second spinal milling cutter 15 is visible, and is arranged concentrically with the axial passage of the first spinal milling cutter 10. This is also evident from the enlarged view of the detail H of FIG. 8 in FIG. 9. FIG. 10 shows the spinal milling cutter system 34 according to FIGS. 6 and 8 in a side view.

FIG. 11 shows the spinal milling cutter system 34 with a second spinal milling cutter 15 completely inserted into the first spinal milling cutter 10 in a perspectival view from an oblique distal direction. The second spinal milling cutter 15, in particular its distal end face 35, protrudes from the distal end face 11 of the first spinal milling cutter 10 by approximately 4 mm, such that the milling cutter toothing 38 and the threaded portion 47 of the second spinal milling cutter 15 are visible. FIG. 12 shows the proximal end portions 31, 49 of both spinal milling cutters 10, 15 of FIG. 11 in an enlarged view. The distal connecting piece part 52 of the connecting piece 50 of the second spinal milling cutter 15 is completely accommodated within the connecting piece 32 of the first spinal milling cutter 10 and is therefore hidden in FIG. 12. A flush transition is formed between the proximal connecting piece part 51 of the second spinal milling cutter 15 and the connecting piece 32 of the first spinal milling cutter 10. As soon as the second spinal milling cutter 15 is set in rotation by a drive, the first spinal milling cutter 10 rotates synchronously with it; the connecting piece 32 of the first spinal milling cutter 10 thus acts as an entrainer to ensure synchronization of the rotational movements of the spinal milling cutters 10, 15. The total length of the spinal milling cutter system 34 is 278 mm, which substantially corresponds to the axial length of the second spinal milling cutter 15.

FIG. 13 shows an enlarged view of the detail A of FIG. 11, the distal end faces 11, 35 of the two spinal milling cutters 10, 15, and their threaded portions 26, 47. As already described, the second spinal milling cutter 15 protrudes axially with a length of approximately 4 mm from the distal end face 11 of the first spinal milling cutter 10. The roundings 12, 36 of the end faces 11, 35 of both spinal milling cutters 10, 15 are clearly visible, as are their tooth profiles 17, 39 and threaded portions 26, 47. Since the outer diameter of the lateral surface 46 of the second spinal milling cutter 15 substantially corresponds to the diameter of the axial passage 14 of the first spinal milling cutter 10, only a comparatively narrow gap is formed between the two spinal milling cutters 10, 15. FIG. 14 shows the representation of FIG. 13 in a side view, from which it can be seen in particular that the angle of inclination and the direction of rotation of the threaded portion 47 of the second spinal milling cutter 15 substantially correspond to the angle of inclination and the direction of rotation of the threaded portion 26 of the first spinal milling cutter 10.

FIG. 15 shows, in particular, the distal end face 11 and the threaded portion 26 of the first spinal milling cutter 10 in a further example, in which the tooth profile 17 of the distal end face 11 also has teeth 18 distributed over the circumference of the distal end face 11 on the radial outer side of the distal end face 11; however, the teeth 18 now point distally, such that the cutting properties of the distal end face 11 of the first spinal milling cutter 10 are improved. The teeth 18 of the tooth profile 17 have, from a distal perspective and seen clockwise, a left, almost right-angled flank 22 and a right flank 23 with an angle of inclination of approximately 40°, with a stepless transition to a recess 24 between two adjacent teeth 18. The tooth profile 17 extends proximally in such a way that, in the example of FIG. 15, the threaded portion 26 is also formed according to the example of FIG. 13, wherein this is effected via an axially longer, stepless transition region 56. This is also evident from the side view of FIG. 16. The spinal milling cutter system 34 shown therein has the same second spinal milling cutter 15 as in FIG. 13; this also protrudes over an axial length of approximately 4 mm beyond the distal end face 11 of the first spinal milling cutter 10.

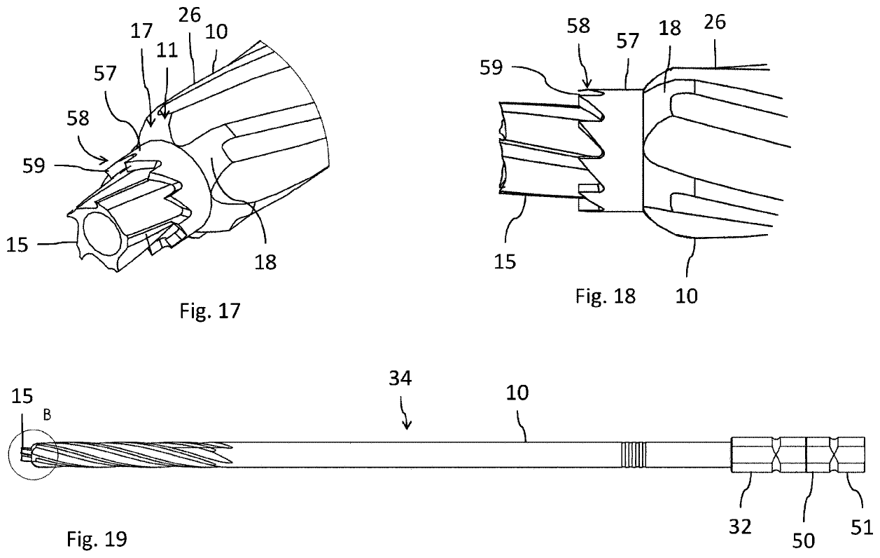

FIG. 17 shows a further example of the distal end face 11 of the first spinal milling cutter 10, which, similarly to the example of FIG. 13, has a tooth profile 17 with radially outward-pointing teeth 18, which, as already described, extend proximally into the threaded portion 16. In contrast to the example of FIG. 13, the distal end face 11 has a distal projection 57 which is provided with a further tooth profile 58 which substantially corresponds to the example according to FIG. 15—in particular, has distally pointing teeth 59. By forming two tooth profiles 17, 58, the example of FIG. 17 therefore combines the cutting ability of the radially outward-pointing teeth 18 according to the example of FIG. 13 with the cutting ability of the distally facing teeth 18 according to the example of FIG. 15. As in the example of FIG. 15, the second spinal milling cutter 15 protrudes distally over a length of 4 mm from the distal end face 11 of the first spinal milling cutter 10, which is also evident from the side view of FIG. 18.

FIG. 19 shows the spinal milling cutter system 34 of FIG. 11 in a side view, wherein the detail B shown there is shown in the three, already described examples by FIGS. 14, 16, and 18.

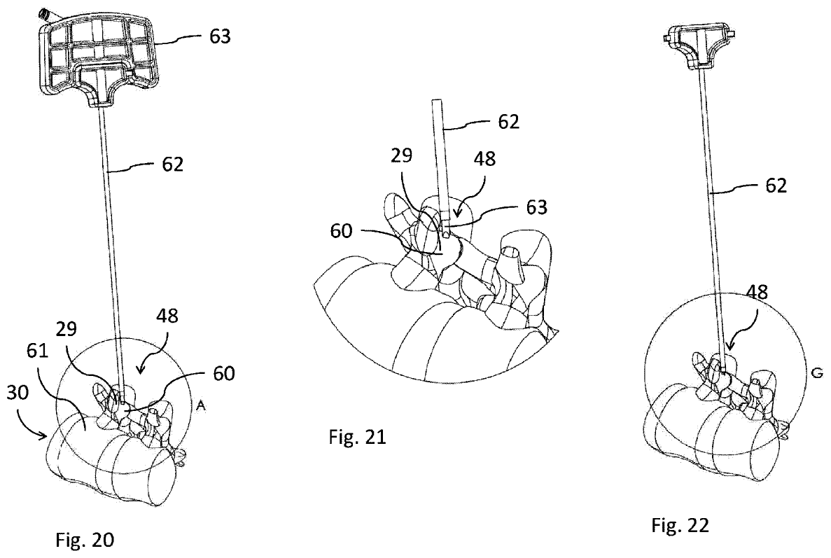

FIGS. 20 to 33 illustrate the sequence of a method according to the invention, in which bone structures 29 in the region of the vertebral column 30 are worked on at a site of use 48, in particular via the spinal milling cutter 10 according to the invention.

FIG. 20 shows an overview of the substantially horizontally oriented vertebral column 30 with upwardly oriented articular processes 60 of the vertebral bodies 61, whose bone structures 29 correspond to the site of use 48. FIG. 21 shows the site of use 29 in an enlarged view of the detail A of FIG. 20. First, a Jamshidi needle 62 shown in FIGS. 20 and 21 is inserted into the site of use 48, the upwardly directed articular process 60 (superior articular process), via a trocar 63 guided within the Jamshidi needle 62.

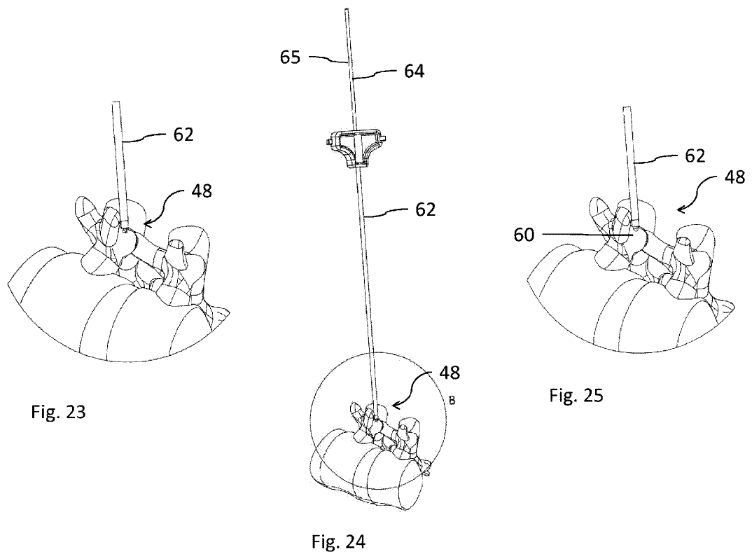

In the transition from FIG. 20 to FIG. 22, the trocar 63 guided within the Jamshidi needle 62 is pulled proximally out of the Jamshidi needle 62. The Jamshidi needle 62 remains at the site of use 48. FIG. 23 shows the site of use 48 with the Jamshidi needle 62 arranged there, in an enlarged view of the detail G of FIG. 22. In the sense of the invention, the Jamshidi needle 62 serves as an access in the present method, since it provides access to the site of use 48 for further instruments for working on the bone structure 29.

In the transition from FIG. 22 to FIG. 24, an awl 64 is guided from distal to proximal through the cavity of the Jamshidi needle 62 to the site of use 48. The awl 64 is then driven into the upwardly directed articular process 60, such that the awl 64 is connected to it. This is also illustrated in FIG. 25 by the view, there shown enlarged, of the detail B of FIG. 24. For the present method, the awl 64 serves as a guide 65 for the insertion of the spinal milling cutter 10 according to the invention, as described below.

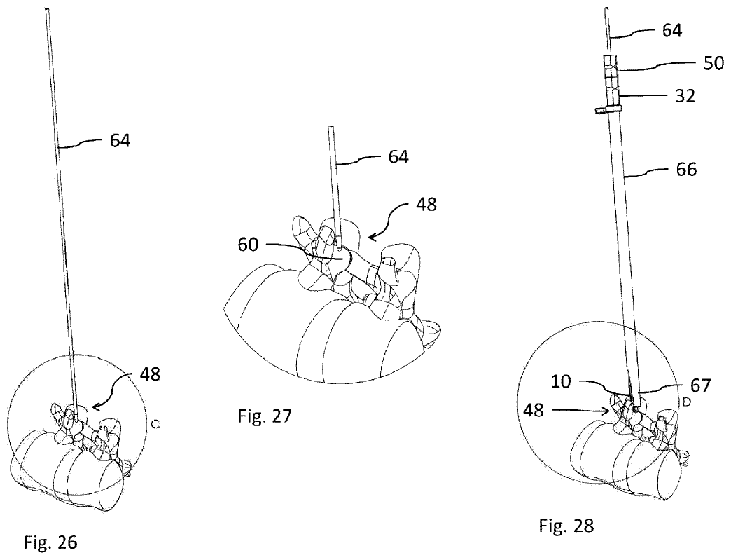

In the transition from FIG. 24 to FIG. 26, the Jamshidi needle 62, as an access, is removed proximally from the site of use 48, while the awl 64 remains as a guide 65 at the site of use 48 and is connected there to the articular process 60. FIG. 27 illustrates the detail C of FIG. 26 in an enlarged view.

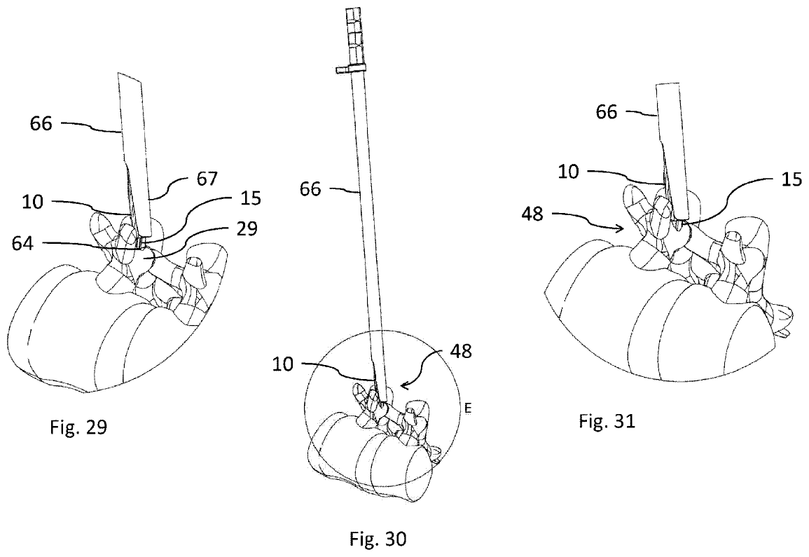

In the transition from FIG. 26 to FIG. 28, a working sleeve 66 with a long lip 67 arranged on the right in FIG. 28 as well as a spinal milling cutter 10 according to the invention and a further spinal milling cutter 15 are pushed over the awl 64 distally to the site of use 48. These components are part of the spinal milling cutter system 34. The working sleeve 66 radially surrounds the first spinal milling cutter 10 according to the invention, which in turn radially surrounds the further, second spinal milling cutter 15. From the positions of the connecting piece 32 of the first spinal milling cutter 10 and the handpiece 50 of the second spinal milling cutter 15 in FIG. 28, it can be seen that the spinal milling cutters 10, 15 are connected to each other in a torque-proof manner. The spinal milling cutters 10, 15 are driven together in rotation, such that the bone structure 29 is worked on at the site of use 48 in such a way that it is first worked on by the second spinal milling cutter 15, which projects distally beyond the first spinal milling cutter 10, until finally the bone structure 29 is also worked on by the first spinal milling cutter 10. During this work, the awl 64 is still connected to the articular process 60. This is also evident from the enlarged view of the detail D of FIG. 28 in FIG. 29.

After the first spinal milling cutter 10 has a sufficiently strong hold in the bone structure 29, the awl 64 is removed proximally from the site of use 48 in the transition from FIG. 28 to FIG. 30. The working sleeve 66 remains at the site of use together with the spinal milling cutters 10, 15, which is also illustrated by the enlarged view of the detail E of FIG. 30 in FIG. 31.

Subsequently, in an optional step, a blunt awl, a guide wire, or a guide rod can be moved distally within the axial passage 55 of the second spinal milling cutter 15 to the site of use 48, and then further work on the bone structure 29 can be carried out by the two spinal milling cutters 10, 15.

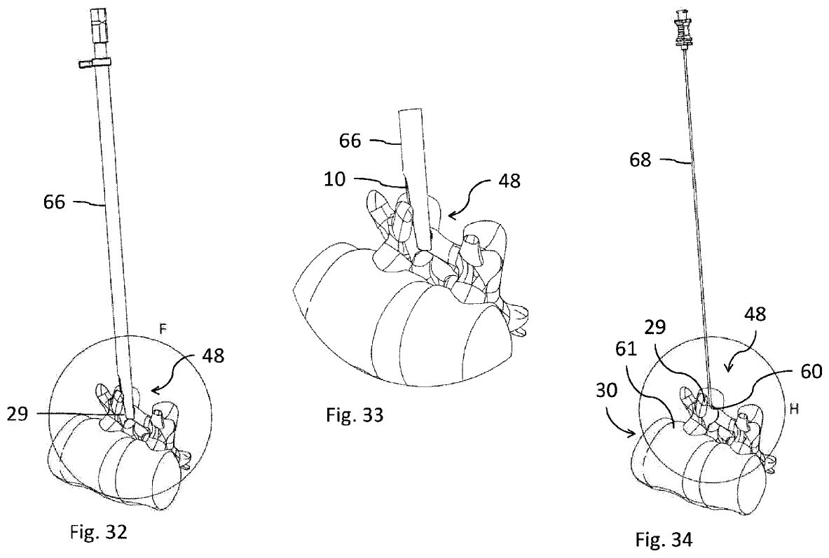

In the transition from FIG. 30 to FIG. 32, the second spinal milling cutter 15, relative to the first spinal milling cutter 10, is moved proximally, first removed from the site of use 48, and finally removed from the first spinal milling cutter 10, such that only the first spinal milling cutter 10, still accommodated in the working sleeve 66, remains at the site of use 48. Thereafter, the bone structure 29 is worked on only via the first spinal milling cutter 10, which is protected by the working sleeve 66 surrounding it. This is also evident from FIG. 33, which shows an enlarged view of the detail F of FIG. 32.

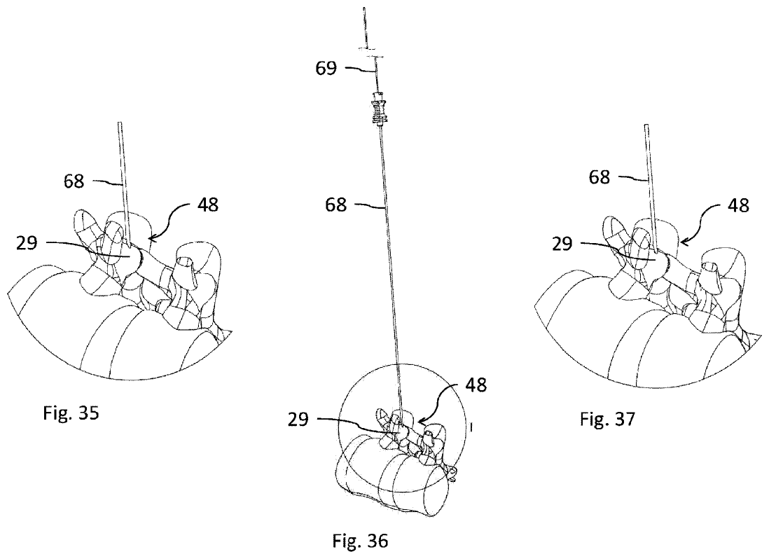

FIGS. 34 to 45 show a further example of the method according to the invention. FIG. 34 shows the site of use 48 similarly to FIG. 20. There, a hollow needle 68, which can also be referred to as an access needle in the sense of an access, is in contact with the bone structure 29 at the site of use 48. The hollow needle 68 has previously been introduced into the site of use 48 using a stylet. FIG. 35 shows an enlarged view of the detail H of FIG. 34, from which the arrangement of the hollow needle 68 on the bone structure 29 can be seen.

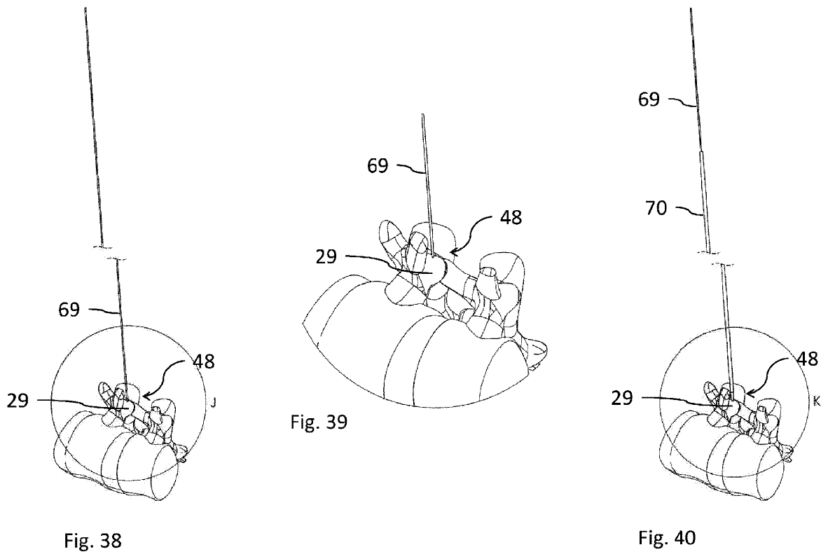

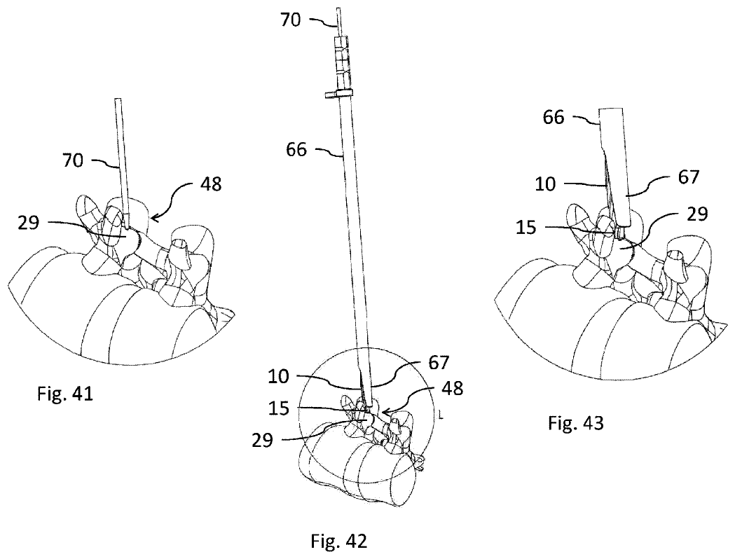

In the transition from FIG. 34 to FIG. 36, after removal of the stylet, a guide wire 69 is brought from proximal to distal through the hollow needle 68 to the bone structure 29 at the site of use 48. This is also evident from the enlarged view of the detail I of FIG. 36 in FIG. 37. In the transition from FIG. 36 to FIG. 38, the hollow needle 68 is removed proximally from the site of use 48, such that only the guide wire 69 remains there—which is also shown in the enlarged view of the detail J of FIG. 38 in FIG. 39. Thereafter, in the transition from FIG. 38 to FIG. 40, a guide rod 70 is moved over the guide wire 69 distally to the site of use 48. FIG. 41 shows an enlarged view of the detail K of FIG. 40.

In the transition from FIG. 40 to FIG. 42, similarly to FIG. 28, the working sleeve 66, including its extended lip 67, and together with the spinal milling cutters 10, 15 accommodated therein, is pushed distally over the guide rod 70 to the site of use 48. FIG. 41 shows an enlarged view of the detail L of FIG. 40.

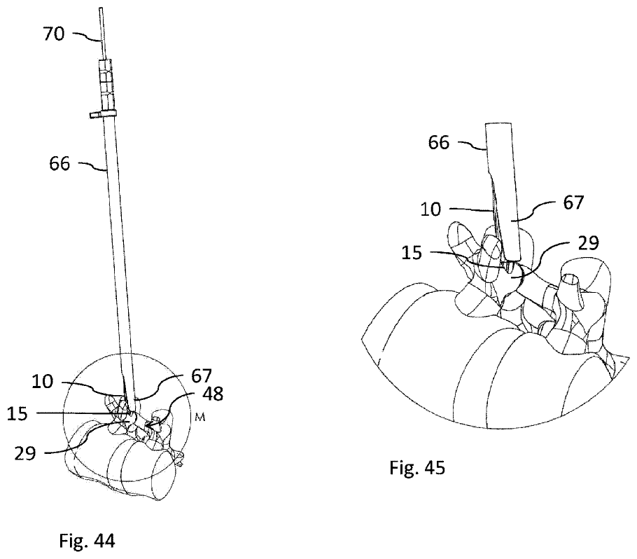

In the transition from FIG. 42 to FIG. 44, as already described in connection with the first example of the method according to the invention, the two spinal milling cutters 10, 15 are driven to rotate and work on the bone structure 29 at the site of use 48 until the first spinal milling cutter 10 has sufficient hold in the bone structure 29. FIG. 45 shows an enlarged view of the detail M of FIG. 44. After the first spinal milling cutter 10 has a firm hold in the bone structure 29, the second spinal milling cutter 15 is removed in the proximal direction from the site of use 48, such that the bone structure 29 is further worked on after this point only with the first spinal milling cutter 10 protected by the working sleeve 66, until the first spinal milling cutter 10 is finally removed from the site of use 48.

The invention being thus described, it will be obvious that the same may be varied in many ways. Such variations are not to be regarded as a departure from the spirit and scope of the invention, and all such modifications as would be obvious to one skilled in the art are to be included within the scope of the following claims.

What is claimed is:

1. A spinal milling cutter for working on bone structures in a region of a vertebral column, the spinal milling cutter comprising:
a distal end face provided with a milling cutter toothing having a tooth profile with at least two teeth; and
an axial passage to receive a further spinal milling cutter.

2. The spinal milling cutter according to claim 1, wherein the tooth profile is designed at least in portions as a cutting edge, or wherein the distal end face of the spinal milling cutter has a rounding that is convex, wherein at least one tooth of the tooth profile is oriented distally and/or radially.

3. The spinal milling cutter according to claim 2, wherein at least one tooth of the tooth profile is not designed to be mirror-symmetric, wherein the tooth has two radial elevations with different radial heights, and wherein a step-like transition is formed between radial elevations of a tooth.

4. The spinal milling cutter according to claim 1, wherein at least one tooth of the tooth profile has a first flank and a second flank, and wherein an inclination of the first flank is greater than an inclination of the second flank, or wherein the tooth profile has at least one tooth with a triangular basic shape.

5. The spinal milling cutter according to claim 1, wherein two adjacent teeth of the tooth profile are separated from each other by a parabolic recess.

6. The spinal milling cutter according to claim 1, wherein the spinal milling cutter has a threaded portion proximal to the milling cutter toothing.

7. The spinal milling cutter according to claim 6, wherein the tooth profile extends proximally such that the threaded portion is formed thereby.

8. The spinal milling cutter according to claim 6, wherein the threaded portion has an axial length between 10 mm and 90 mm, or between 20 mm and 75 mm, or is approximately 60 mm, or wherein the threaded portion has a thread pitch between 60° and 85°, or between 70° and 85°, or between 75° and 80°, or between 75° or 80°.

9. The spinal milling cutter according to claim 1, wherein the distal end face of the spinal milling cutter has a distal projection, and wherein the projection is provided with a further tooth profile.

10. The spinal milling cutter according to claim 9, wherein the tooth profile of the projection is designed to be mirror-symmetric, and wherein the tooth profile of the projection have teeth having a triangular basic shape.

11. The spinal milling cutter according to claim 1, wherein a proximal end portion of the spinal milling cutter has a connecting piece to receive the further spinal milling cutter.

12. A spinal milling cutter system comprising:
the first spinal milling cutter according to claim 1; and
a second spinal milling cutter adapted to be accommodated as a further spinal milling cutter in the axial passage of the first spinal milling cutter, and which has a distal end face with a milling cutter toothing having a tooth profile.

13. The spinal milling cutter system according to claim 12, wherein the second spinal milling cutter is adapted to be connected to the first spinal milling cutter in a torque-proof and/or detachable manner, wherein the second spinal milling cutter has a proximal connecting piece whose outer contour is designed at least in portions to be complementary to the inner contour of the connecting piece of the first spinal milling cutter.

14. The spinal milling cutter system according to claim 12, wherein the spinal milling cutters are adapted to rotate independently of one another.

15. The spinal milling cutter system according to claim 12, wherein the second spinal milling cutter has an axial passage which, when connected to the first spinal milling cutter, is arranged concentrically with the axial passage of the first spinal milling cutter.

16. The spinal milling cutter system according to claim 12, wherein the tooth profile of the second spinal milling cutter extends proximally such that a threaded portion of the second spinal milling cutter is formed.

17. A method for working on bone structures in a region of the vertebral column at a site of use, the method comprising:
providing the spinal milling cutter according to claim 1;
inserting the spinal milling cutter with a further spinal milling cutter accommodated in the axial passage of the spinal milling cutter along a guide towards the site of use, the guide being arranged within the axial passage of the spinal milling cutter within the axial passage of the further spinal milling cutter;
rotating the first spinal milling cutter together with the further spinal milling cutter such that the bone structure at the site of use is worked on along a predefined path;
removing the further spinal milling cutter from the site of use;
rotating the first spinal milling cutter such that the bone structure is worked on in the desired manner at the site of use; and
removing the first spinal milling cutter from the site of use.

18. The method according to claim 17, wherein the guide is connected to the bone structure before the spinal milling cutters are inserted into the site of use, or wherein, before the spinal milling cutters are inserted into the site of use after the guide has been connected to the bone structure, a protective sleeve is pushed over the guide or over the first spinal milling cutter towards the site of use.

19. The method according to claim 17, wherein the work on the bone structure by the first spinal milling cutter is carried out substantially by its lateral surface.

20. The method according to claim 17, wherein access to the site of use is via an articular process of a vertebral body via an upwardly directed articular process of the vertebral body.
